(19) United States
(12) Patent Application Publication
(10) Pub. No.: US 2026/0144940 A1
(43) Pub. Date: May 28, 2026

(54) MEDICAL NEEDLE

(71) Applicant: Becton, Dickinson and Company, Franklin Lakes, NJ (US)

(72) Inventors: Srinath B., Bengaluru (IN); Shishir Prasad, Ramsey, NJ (US); Karthik MR, Bengaluru (IN); Radhika Dharmadhikari, Pune (IN)

(73) Assignee: Becton, Dickinson and Company, Franklin Lakes, NJ (US)

(21) Appl. No.: 18/958,323

(22) Filed: Nov. 25, 2024

(51) Int. Cl. A61M 5/34; A61M 5/31; A61M 5/32

(52) U.S. Cl. CPC A61M 5/343; A61M 5/3134; A61M 5/3234

(57) ABSTRACT

A medical needle apparatus isolates external deflectional loads applied to it after its needle cannula is advanced along a penetration pathway into a selected penetration site of a patient, for example for administering medicinal fluids via a syringe or for performing biopsies. The needle apparatus includes an elongated needle cannula, and a needle hub coupled to a proximal first end of the needle cannula. In some embodiments, a stabilization patch circumscribes the needle cannula. The stabilization patch includes a contact surface on its distal end for abutting anchoring contact on the patient's body. The anchored stabilization patch resists external deflectional loads so that the needle cannula remains stationary at the penetration site. In other embodiments, the needle hub incorporates a flexible hub joint that deflects upon application of external loads while the needle cannula remains stationary. Other embodiments incorporate both a stabilization patch and a flexible hub joint.

### TECHNICAL FIELD

The present disclosure generally relates to medical needle apparatus for advancement into a selected penetration site of a patient for performing a medical procedure. More particularly, embodiments of the present disclosure relate to medical needles that isolate external loads applied to the extracorporeal portion of a needle apparatus so that those loads are not transferred to the intracorporeal portion of the needle cannula that is within the patient's body.

### BACKGROUND

Various clinical procedures require deep insertion of relatively long, rigid medical needles into patients for lengthy treatment time periods. Exemplary clinical procedures include spinal anesthetization for Cesarean delivery, orthopedic surgeries, hernias and vascular surgery, percutaneous needle biopsies, percutaneous ethanol injection (PEI) treatments for thyroid and liver cancers, and long-term indwelling injections. Inadvertent movement of those long, rigid needles, by application of external lateral loads relative to the needle central axis may injure body tissue within the unintended movement path and/or translate the needle tip out of an intended penetration site. During the medical procedure, if the needle is dispensing drugs or other medicinal fluids to the patient during inadvertent needle movement, they may not be dispensed to the intended delivery site. If the needle is moved during a biopsy procedure, the extracted tissue sample might not be from the intended site. Unintended consequences of needle deflection are not limited to body cavity specific procedures with long, rigid medical needles. When typical flexible, small-gauge, short injection needles are used to administer low injection rate, sub-dermal, subcutaneous or intra-muscular injections of less than 10 seconds/mL, they are also susceptible to inadvertent needle movement. Exemplary low injection rate medications include insulin, blood thinners, fertility drugs, epinephrine, biologics and monoclonal antibodies, as well as medications for pain relief, autoimmune diseases, and palliative care.

FIG. 1 depicts a patient 30 undergoing intracorporeal treatment to the L2 region of spinal column 32. As part of the procedure, the patient's interspinous ligament 34 requires a subcutaneous injection through the skin 36 at a needle entry point 36 to chosen injection site or penetration site 40. The terms injection site and penetration site are used interchangeably in this disclosure. Generally, a penetration site designates needle tip placement at a location for further performance of any procedure (e.g., for biopsy extraction of tissue or dispensing of a medicinal fluid); an injection site implies delivery of a drug or other medicinal fluid to the patient via the needle. A known needle apparatus 42 includes a needle cannula 44 with a needle tip 45 and a needle hub 46. A known syringe 48 is coupled to a needle connector of needle hub 46. The subcutaneous portion of needle cannula 44 is oriented along a penetration pathway between the needle entry point 38 and the injection or penetration site 40. The needle apparatus 42 is advanced or retracted along the penetration pathway by application of an axial load force FA. Any non-axial, transverse, loads Fr applied to the known needle hub 46 or the syringe 48 deflects the external, exposed portion of the needle apparatus 42 as shown in the phantom displacement positions. The transverse loads also deflect the subcutaneous portion of the needle apparatus 42. As shown in FIG. 1, deflection of the subcutaneous portion of the needle cannula 44 moves its needle tip 45 out it the penetration site within the interspinous ligament 34. Displacement of the needle tip 45 outside of the intended penetration site 40 will not facilitate the intended delivery of medication to the interspinous ligament 34 and the syringe. Needle cannula 44 displacement may cause internal tissue damage within its actual displacement path.

The likelihood of unintended needle apparatus 42 displacement is exacerbated by needle length and/or its deflectional rigidity. Analogizing the needle entry point 38 as a lever fulcrum, increasing needle cannula 44 penetration depth increases the potential displacement arc of the needle tip 45 within the patient. Similarly, increasing needle cannula 44 and/or syringe 48 axial length increases the torque moment exerted on the needle apparatus 42. Relatively stiffer needle cannulas increase the possibility of more needle tip arcuate gross motion as the cannula deflects rather than bends in response to applied transverse loads. Medical procedure elapsed time length while the needle apparatus 42 remains inserted within the patient 30 increases likelihood of inadvertent needle apparatus 42 deflection; whether caused by administering healthcare professionals or by patient reflex movement actions. deflection.

### SUMMARY

A medical needle apparatus isolates external deflectional loads applied to it after its needle cannula is advanced along a penetration pathway into a selected penetration site of a patient; for example, for administering medicinal fluids via a syringe or for performing biopsies at the penetration site. Stable orientation of the inserted needle cannula within the patient's body through an entire procedure enhances patient comfort, better maintains needle positioning within the intended penetration site for delivery of medications or extraction of tissue samples and reduces likelihood of potential damage to tissue surrounding the penetration site caused by unintended needle deflection. The needle apparatus includes an elongated needle cannula, and a needle hub coupled to a proximal first end of the needle cannula. In some embodiments, a stabilization patch circumscribes the needle cannula. The stabilization patch includes a contact surface on its distal end for abutting anchoring contact on the patient's body. The anchored stabilization patch resists external deflectional loads so that the needle cannula remains stationary at the penetration site. In other embodiments, the needle hub incorporates a flexible hub joint that deflects upon application of external loads while the needle cannula remains stationary. Yet other embodiments incorporate both a stabilization patch and a flexible hub joint.

One aspect of the present disclosure pertains to a medical needle apparatus including an elongated needle cannula defining a first central axis through a first lumen formed therein. The needle cannula has a proximal first end, and a distal first tip for advancement into the body of a patient. The needle apparatus also includes a needle hub defining a second central axis through a second lumen formed within it. The needle hub has a distal second tip that is coupled to the first end of the needle cannula and a proximal second end, which defines an open, exposed hub cavity that is in common fluid communication with the first and second lumens. The needle apparatus also includes a stabilization patch defining a third central axis through a third lumen formed within it. The third lumen circumscribes an outer circumferential surface of the needle cannula. The stabilization patch has a contact surface on a distal third tip thereof for abutting contact with a selected external anchoring site of a patient's body. The anchoring site surrounds a needle entry point. The entry point initiates a penetration pathway of the needle cannula that terminates at a selected needle penetration site within the patient. The stabilization patch has a proximal, third end facing the second end of the needle hub, and an axial length between its third axial tip and its third axial end that is less than a corresponding axial length of the needle cannula. The stabilization patch is selectively slidable bidirectionally along the needle cannula from the first distal tip towards the second end of the needle hub. The respective first, second and third central axes are mutually coaxial with each other.

Another aspect of the present disclosure pertains to a method for treating a patient by advancing the above-described medical needle apparatus into a selected needle penetration site within a patient. The needle apparatus is used in patient treatment by selecting a needle penetration site of a patient. After site selection, the first tip and first central axis of the needle cannula are oriented at a needle entry point, which initiates a penetration pathway of the needle cannula within the patient that terminates at the selected penetration site. The needle cannula is advanced into the patient, along the penetration pathway, to orient the first tip within the selected penetration site. After cannula penetration to the penetration site, the contact surface of the stabilization patch is advanced by sliding motion into abutting contact with the patient's body at an anchoring site surrounding the needle entry point. This contact couples the stabilization patch to patient's body, and when so coupled the stabilization patch resists any external deflectional loads applied to the needle apparatus, maintaining stable orientation of the needle cannula at the penetration site. In some embodiments, the stabilization patch allows axially oriented displacement of the needle apparatus relative to its respective central axes during the patient medical treatment.

Yet another aspect of the present disclosure pertains to a medical needle apparatus, including an elongated needle cannula defining a first central axis through a first lumen formed therein, a proximal first end, and a distal first tip for advancement into a body of a patient. The needle apparatus also includes a flexible needle hub defining a second central axis through a second lumen formed within it. A distal second tip of the hub is coupled to the first end of the needle cannula and a proximal second end of the hub defines an open, exposed hub cavity that is in common fluid communication with the first and second lumens. In some embodiments, the exposed hub cavity forms a Luer connector for coupling to a vascular device, such as a syringe. The first and second central axes are coaxial with each other. A flexible hub joint is interposed between and coupled to the second tip and to the second end of the hub. When the needle apparatus is advanced into a patient, the flexible hub joint transfers loads applied to the needle assembly that are aligned with the coaxial central axes, while the flexible hub joint isolates non-axially oriented deflection loads applied to the second end without deflecting the second tip or the needle cannula. In this way, the needle cannula remains stationary at the penetration site within the patient, despite application of external deflectional loads on the second end of the hub.

In some aspects of the present disclosure, the needle apparatus includes both a stabilization patch and a flexible hub joint. In some embodiments, the stabilization patch also incorporates a flexible stabilization patch joint. In some embodiments, the needle apparatus is an introducer needle. In other embodiments the needle apparatus is a spinal needle or an injection needle.

### BRIEF DESCRIPTION OF THE DRAWINGS

Exemplary embodiments of the disclosure are further described in the following detailed description in conjunction with the accompanying drawings, in which:

FIG. 1 depicts a known medical needle and syringe, where the needle tip is advanced within a penetration site within an interspinous ligament of a patient;

FIG. 2 is an isometric view of an embodiment of a needle apparatus of the present disclosure with its stabilization patch in abutting contact with the patient's body at an anchoring site;

FIG. 3 is an isometric view of the needle apparatus of FIG. 2 after withdrawal of the stabilization patch from the anchoring site on the patient;

FIG. 4 is an axial cross-sectional view of the needle apparatus of FIG. 3;

FIGS. 5-7 are isometric views embodiments of stabilization patches of the present disclosure;

FIG. 8 is an elevational view of an embodiment of a needle apparatus of the present disclosure, shown in partial cross section, having a flexible hub joint, with an axial load applied to the needle hub;

FIG. 9 an elevational view of the needle apparatus of FIG. 8, shown in partial cross section, with a non-axial load applied to the needle hub:

FIG. 10 is an isometric view of the deflected flexible hub joint of FIG. 9;

FIGS. 11 and 12 are elevational views of an embodiment of a needle apparatus of the present disclosure, shown in partial cross section, having a flexible hub joint with a slidable hub collar;

FIG. 13 is an isometric view of an embodiment of a needle apparatus of the present disclosure with both a flexible hub joint and a flexible stabilization patch joint and respective slidable, dedicated hub and stabilization patch collars;

FIGS. 14 and 15 show the needle apparatus of FIG. 13 during affixation of the flexible stabilization patch to an anchoring site of a patient, and activation of the stabilization patch collar to stiffen the stabilization patch;

FIGS. 16-18 are an alternative embodiment of the needle apparatus of FIG. 13, shown in partial cross section, wherein the flexible collar and the flexible stabilization patch share a common sleeve collar;

FIG. 19 is an embodiment of a flexible stabilization patch having a ball-and-socket joint, with the joint aligned axially with the central axis of the needle cannula;

FIG. 20 is an embodiment of a flexible stabilization patch having a ball-and-socket joint, with the joint deflected at an angle a relative to the central axis of the needle cannula; and

FIGS. 21-23 are schematic representations of the ball-and-socket joint being selectively deflected and locked into a desired deflection angle a relative to the central axis of the needle cannula, shown in partial cross section.

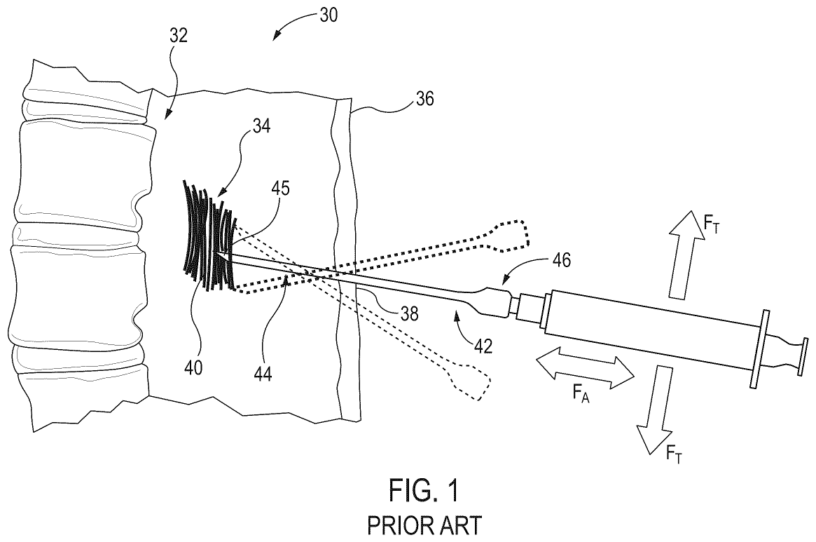

To facilitate understanding, identical reference numerals have been used, where possible, to designate identical elements that are common to the figures. The figures are not drawn to scale.

### DETAILED DESCRIPTION

A medical needle apparatus isolates external deflectional loads applied to it after its needle cannula is advanced along a penetration pathway into a selected penetration site of a patient, for example for administering and delivering medicinal fluids via a syringe or for performing biopsies. The needle apparatus includes an elongated needle cannula, and a needle hub coupled to a proximal first end of the needle cannula. In some embodiments, a stabilization patch circumscribes the needle cannula. The stabilization patch includes a contact surface on its distal end for abutting anchoring contact on the patient's body. The anchored stabilization patch resists external deflectional loads so that the needle cannula remains stationary at the penetration site. In other embodiments, the needle hub incorporates a flexible hub joint that deflects upon application of external loads while the needle cannula remains stationary. Other embodiments incorporate both a stabilization patch and a flexible hub joint. Isolation of external deflectional loads on the needle hub maintains stable orientation of the needle cannula within the patient. Stable orientation of the inserted needle cannula within the patient's body through an entire procedure enhances patient comfort, better maintains needle positioning within the intended penetration site for delivery of medications or extraction of tissue samples and reduces likelihood of potential damage to tissue surrounding the penetration site caused by unintended needle deflection.

In this disclosure, a convention is followed wherein the distal end of the device is the end closest to a patient, e.g., for delivery of one or more drugs to the patient, and the proximal end of the device is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, I.V. tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a syringe discharge tip and can interlock and connect to the female end that is incorporated within a proximal end of a needle hub of a drug dispensing needle or filling needle for aspirating a drug from a drug vial into a syringe. Luer connector female ends are commonly located on vascular access devices (VADs).

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “wall”, “top”, “side”, “bottom” and others are used throughout this specification to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

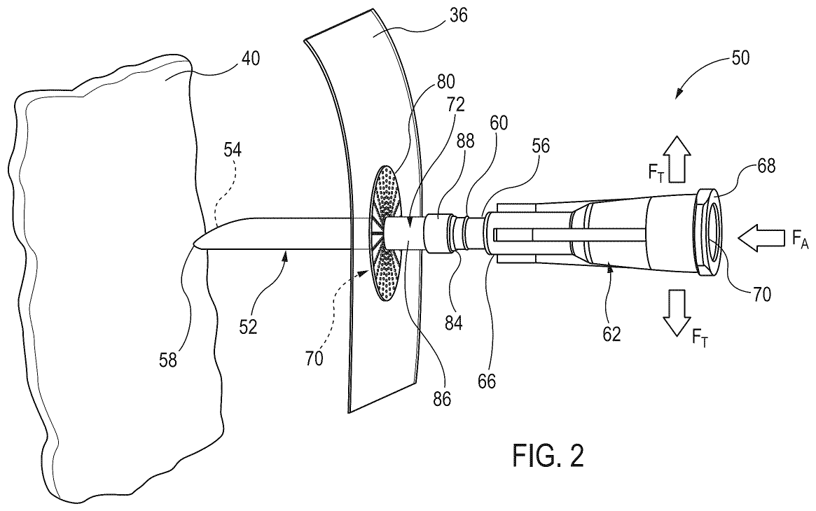

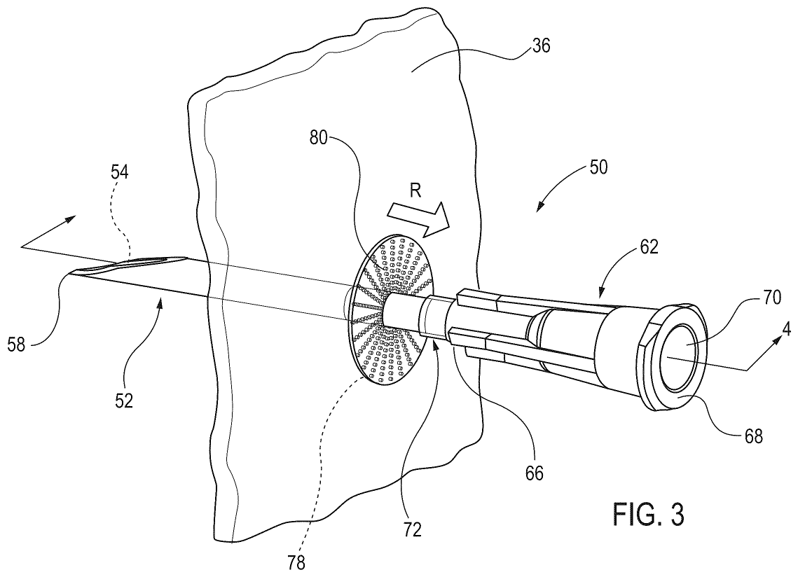

The following non-limiting examples demonstrate principles according to one or more embodiments of the disclosure. FIGS. 2-5 depict a medical needle apparatus with an external stabilization pateh that isolates external, non-axial displacement loads from the inserted portion of its needle cannula. The needle apparatus 50 includes an elongated needle cannula 52 that defines a first central axis through a first lumen 54 that is formed therein. The needle cannula 52 includes a proximal first end 56, a distal first tip 58 and a ridge 60 that extends radially from the outer circumference of the cannula. In some embodiments, the ridge 60 is integrally formed within the needle cannula, or is a separate component that is affixed to the needle cannula (e.g., an O-ring or a circumferentially wound strip. In other embodiments, the needle cannula does not have the ridge 60. As shown in those figures, distal first tip 58 of the needle cannula 52 is advanced through the skin 36 of a patient at a needle entry point 38 to a selected penetration site 40, along an injection path defined between them.

The needle apparatus 50 includes needle hub 62 that defines a second central axis through a second lumen 64 formed therein. A distal second tip 66 of the needle hub 62 is coupled to the first end 56 of the needle cannula 52. A proximal second end 68 of the needle hub 62 defines an open, exposed hub cavity 70 that is in common fluid communication with the first 54 and second 64 lumens. In this embodiment of FIGS. 2-4 the hub cavity 70 is a Luer profile fitting for receipt of a tip of a syringe. In other embodiments the hub cavity is configured to couple to various other types of medical devices, including vascular access devices (VADs).

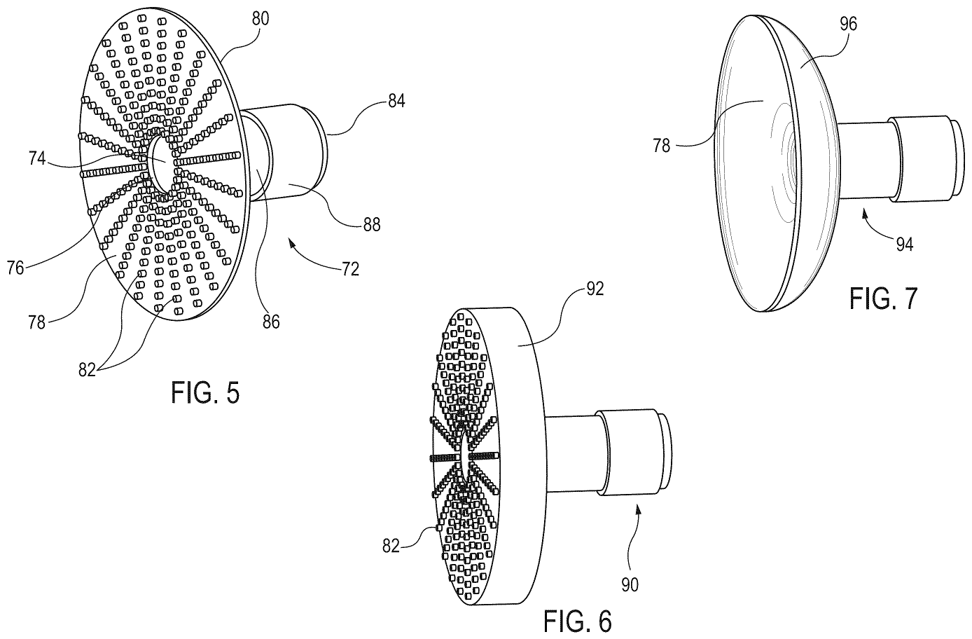

The needle apparatus 50 includes a stabilization patch 72 that defines a third central axis through a third lumen 74 formed therein and a distal tip 76. The third lumen 74 circumscribes an outer circumferential surface of the needle cannula 52. The stabilization patch 72 has a contact surface 78 on its distal third tip 76 for abutting contact with a selected external anchoring site of patient's body. In FIGS. 2-4 that anchoring site surrounds the needle entry point 38 on the patient's skin 36, so that it is coincident with the penetration pathway of the needle cannula. That penetration pathway terminates at the needle penetration site 40. Referring also to FIG. 5, the contact surface 78 comprises a pliable skirt 80 that is conformable to the contour of the patient's skin 36. The distal surface of the contact surface 78 incorporates an anchoring media for coupling to the skin surface 36. In FIG. 5, the stabilization patch 72 is constructed of polymer material and the anchoring media comprise molded pins. In other embodiments the anchoring media comprise micro needles, medical grade adhesive or suction cups. The stabilization patch 72 has a proximal, third end 84 that faces the second end 68 of the needle hub 62 and an axial length between its third tip 76 and its third end that is less than a corresponding axial length of the needle cannula 52. The stabilization patch 72 comprises a tubular body 86 with a finger grasping surface 88 that is proximate its third end 84. By manipulating the finger grasping surface 88 the stabilization patch 72 is selectively slidable bi-directionally along the needle cannula 52 from the first distal tip 58 towards the second end 68 of the needle hub 62. Sliding motion of the stabilization patch 72 is arrested when the surface forming the third lumen 74 contacts the external ridge 60 of the needle cannula 52.

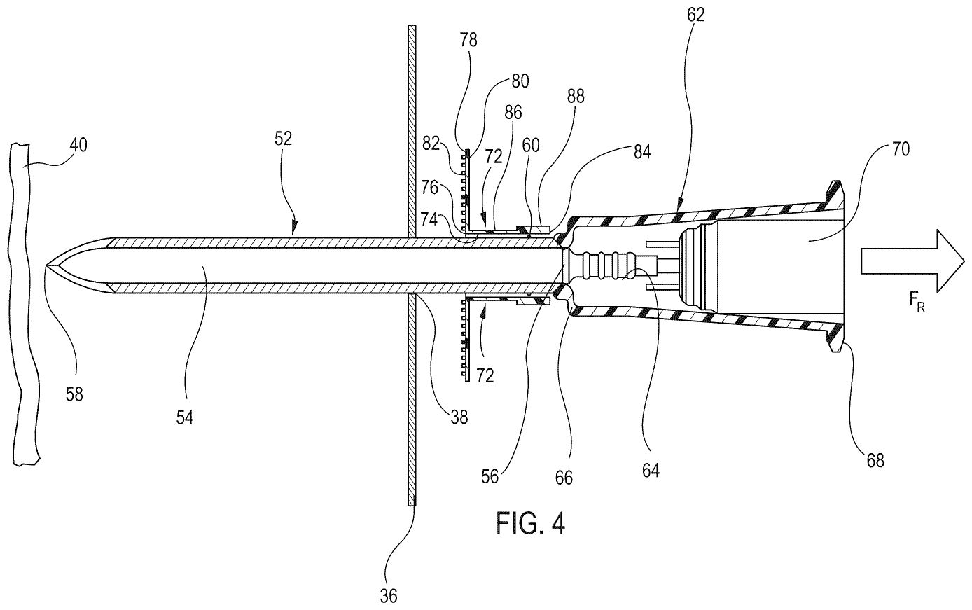

As is shown in FIGS. 2-4, the respective first, second and third central axes or the respective needle cannula 52, needle hub 62 and stabilization patch 72 are mutually coaxial with each other, so that needle advancement axial forces F_{A} that are exerted on the needle apparatus 50 to advance the needle into the patient's body to the penetration site 40 do not deflect it laterally. When the needle apparatus 50 is used for patient treatment, the medical practitioner selects the needle penetration site 40. The first tip 58 and first central axis of the needle cannula 50 are oriented at an external anchoring site of the patient's body. The anchoring site surrounds a needle entry point 38. Needle entry point 38 initiates a penetration pathway of the needle cannula that terminates at the selected penetration site 40, i.e., the linear path between them. The practitioner advances the needle cannula into the patient, along the penetration pathway, to orient the first tip within the selected penetration site 40. After advancement of the needle tip 58 to the penetration site 40, (e.g., after coupling of a syringe or other medical device to the hub cavity 70-not shown), the medical practitioner slides and advances the stabilization patch 72 so that the contact pins 82 on the contact surface 78 of the stabilization patch 72 are in abutting contact with the patient's skin 36. That abutting contact anchors the stabilization patch 72 to the patient's body. Upon completion of the anchoring, the stabilization patch 72 resists non-axial, transverse force loads Fr exerted on the apparatus 50, thereby isolating motion of the portion of the needle cannula 52 that is within the patient's body. When the medical procedure involves dispensing of a medical fluid into the penetration site 40, a syringe tip of a syringe (not shown) is coupled to the Luer connector formed in the hub cavity syringe 70 and the syringe contents is dispensed to the penetration site. After dispensing the syringe contents or completion of any portion of the medical procedure, the needle apparatus 50 is retracted sequentially from the patient by first retracting the stabilization patch 72 to decouple it from the patient's skin 36; then the entire apparatus is retracted axially in the direction F_{R} of FIG. 4. In some procedures, the medical device that is coupled to the syringe cavity 70 is removed prior to removal of the needle apparatus 50 from the patient.

Alternate stabilization patch embodiments are shown in FIGS. 6 and 7. The stabilization patch 90 of FIG. 6 comprises a non-pliable, rigid flange 92, with otherwise the same tubular structure, contact surface 78, and anchoring media molded pins 82 of the stabilization patch 72. In other embodiments, the anchoring media of the rigid flange structure comprise micro needles or medical grade adhesive. The Stabilization patch 94 of FIG. 7 comprises a flexible suction cup skirt 96; its contact surface 78 and contact media comprises the suction cup structure. The stabilization patch 94 otherwise comprises the same tubular structure as the stabilization patch 72. In some embodiments, the stabilization patches 72, 90 and 94 are adapted to be utilized with existing, off-the-shelf needle cannulas and hubs.

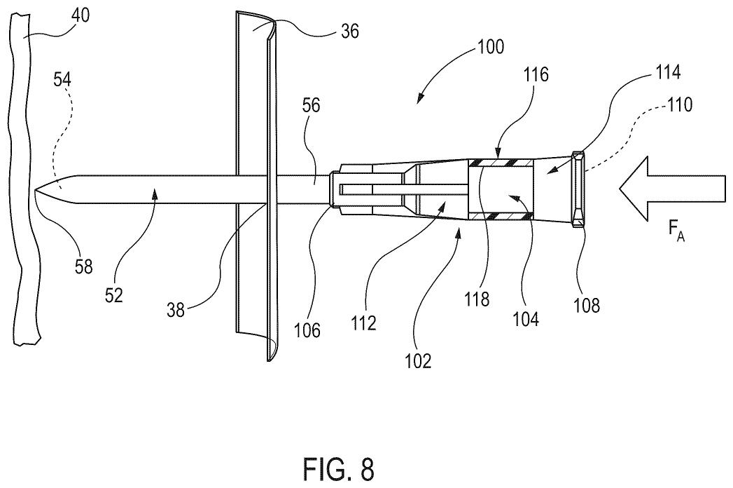

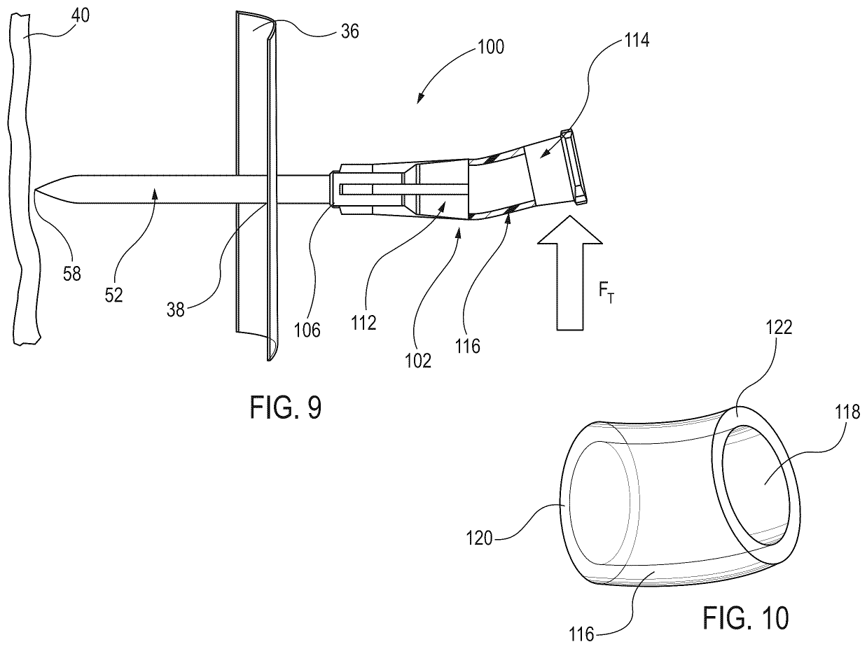

The needle apparatus 100 of FIGS. 8-10 incorporates a multi-piece, flexible needle hub 102 that yields to non-axial, transverse loads applied to it. Hub 102 isolates those applied loads from a distal portion of a needle cannula 52 that is oriented within the patient. Similar to the needle apparatus 50, the needle apparatus 100 includes the elongated needle cannula 52 that defines a first central axis through a first lumen 54 formed therein, a proximal first end 56, and a distal first tip 58 for advancement through skin 36 at an injection entry point 38 into a penetration site 40 within a patient's body.

The multi-piece, flexible needle hub 102 defines a second central axis through a second lumen 104 that is formed therein. The distal second tip 106 of hub 102 is coupled to the first end 56 of the needle cannula 52. The proximal second end 108 of hub 102 defines an open, exposed hub cavity 110 that is in common fluid communication with the first and second lumens. In the embodiment of FIGS. 8-10 the hub cavity 110 is a Luer connector; in other embodiments the hub cavity 110 is configured to couple with other types of medical connectors, including VAD fittings. The multi-piece, flexible hub 102 comprises three coupled segments that are externally sealed, in internal fluid communication with each other by the second lumen 104: distal hub segment 112; proximal hub segment 114; and an intermediate flexible hub joint 116 that is interposed between the distal and proximal hub segments. The flexible hub joint 116 transfers axial loads FA that are applied to the needle assembly, i.e., that are aligned with the coaxial first and second central axes, while it isolates non-axially oriented deflection loads Fr that are applied to the second end 108 of the hub 102, without deflecting the hub's second tip 106 or the needle cannula 52.

As shown in FIG. 10, the flexible hub joint 116 includes a flexible member lumen 118, a distal axial end 120 and a proximal end 122. In some embodiments the hub joint 116 is a memory retention tube formed with polyurethane, silicone, polyvinyl chloride (PVC), polytetrafluoroethylene (PTFE) or thermosetting materials, which will resist applied deflection loads and return to its non-deflected, relaxed, tubular state when those loads are removed. When the flexible hub joint 116 is its relaxed tubular state, the flexible member lumen118 is coaxial with the second central axis of the hub 102. In some embodiments, the flexible hub joint does not have a tubular structure. In some embodiments rigidity of the flexible hub joint 116 is enhanced with axially oriented strips about its circumference.

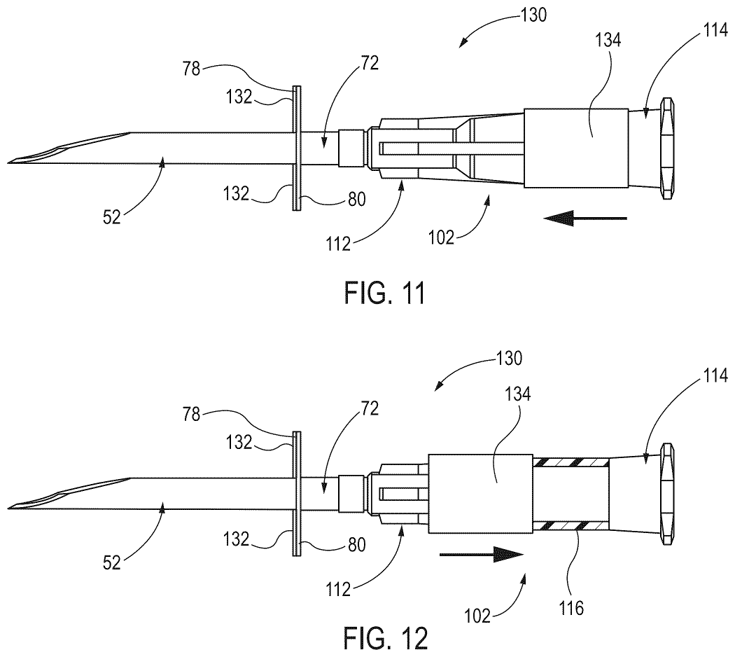

Embodiments of medical needle apparatus of FIGS. 11-23 disclosed herein combine an anchored stabilization patch's inherent stiffness with a flexible hub's ability to bend in response to externally applied deflection forces. The bending response of the flexible hub avoids deflection of the more distal needle cannula, while the anchored stabilization patch generally stiffens the extracorporeal portion of the needle cannula, which in turn isolates the intracorporeal portion of the needle cannula from those externally applied deflection forces.

The needle apparatus 130, shown in FIGS. 11 and 12, incorporate both a stabilization patch 72 and a flexible hub 102, along with the needle cannula 52. The contact surface 78 of the flexible skirt 80 of the stabilization patch 72 utilizes a layer of medical grade adhesive 132 for its anchoring media. A hub collar 132 circumscribes the needle hub 102; it is selectively slidable axially between the distal 112 and proximal 114 hub segments to circumscribe the flexible hub joint 116. When the hub collar 116 is not circumscribing the flexible hub joint 116, the latter is free to flex in response to loads applied to the needle apparatus 130. Conversely, the hub collar 132 stiffens bending resistance of the flexible hub joint 116 when it circumscribes that joint. In practice, the hub collar 132 is useful to minimize flexible hub joint 116 deflection until the needle cannula 52 insertion is completed.

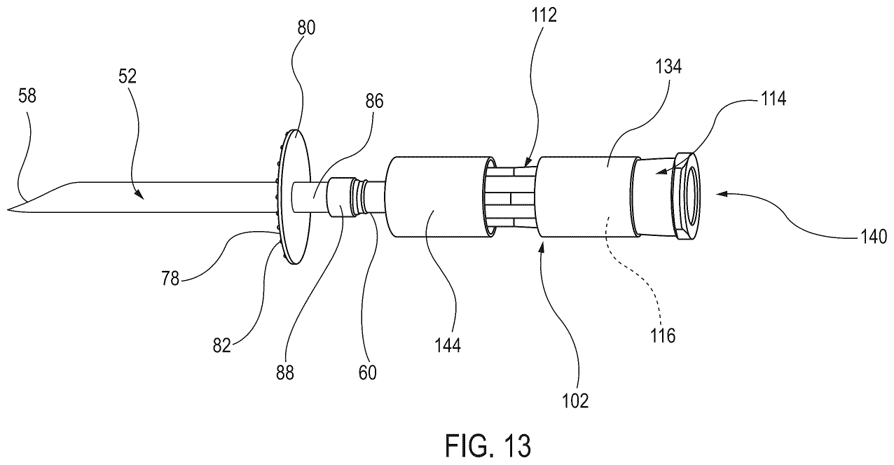

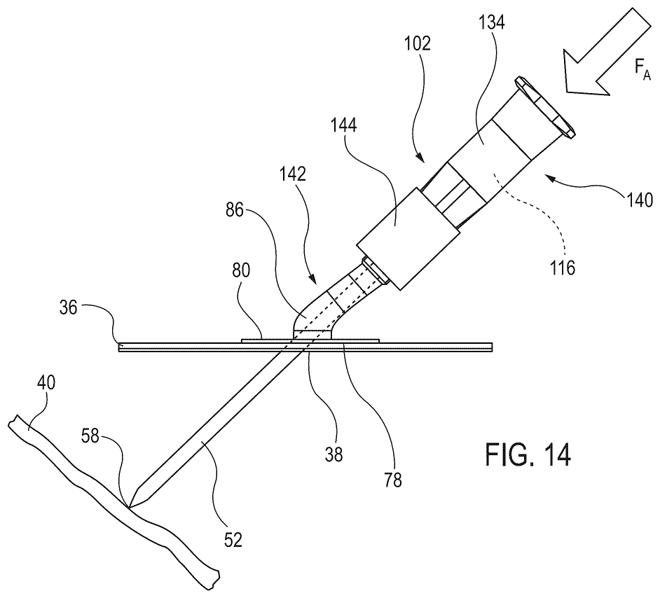

The needle apparatus 140 of FIGS. 13-15 incorporates the flexible hub 102 and collar 134 of the embodiments of FIGS. 11 and 12, in combination with a flexible stabilization patch 142. Needle apparatus 140 is capable of isolating external applied loads even when it has a low-angled entry into the patient's body. The flexible stabilization patch 142 slides over the needle cannula 52 and ridge 60 and it incorporates a flexible tubular body 86, and finger grasping surface 88 of similar structure to those of the corresponding stabilization patch 72. As shown in FIGS. 14 and 15, the flexible stabilization patch 142 conforms to the relative angular orientation of the inserted needle cannula 52 through the patient's skin 36, from the needle entry point 38 of the distal needle tip 58 to the penetration site 40, along the penetration pathway. After needle penetration, the flexible stabilization patch 142 is advanced distally toward the needle entry point 38 so that the contact surface 78 of its flexible skirt 80 and its anchoring media 82 abut against the patient's skin 36. After abutment of the anchoring media 82, a stabilization patch collar 144 is selectively slidable axially to circumscribe the tubular body 86, and finger grasping surface 88 of the flexible stabilization patch 142. The stabilization patch collar 144 stiffens bending resistance of the flexible stabilization patch 142 when it circumscribes the latter.

As previously described, the hub collar 132 circumscribes the needle hub 102; it is selectively slidable axially between the distal 112 and proximal 114 hub segments to circumscribe and stiffen bending resistance of the flexible hub joint 116.

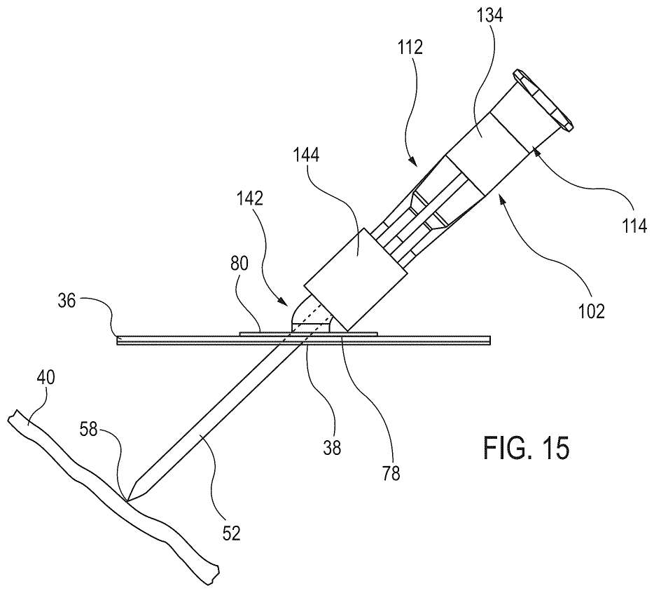

FIGS. 16-18 are an embodiment of a needle apparatus 150 with a single common collar 152 that selectively stiffens either the flexible stabilization patch 142 or the flexible hub joint 116. In FIG. 16, the common collar 152 is in a neutral position that is not stiffening either the flexible stabilization patch 142 or the flexible hub joint 116. In FIG. 17, common collar 152 is retracted over the three segments 112, 114 and 116 of the flexible hub 102 to stiffen the joint for initial advancement and insertion of the needle cannula 52 into the patient. After initial needle cannula 52 insertion and orientation of the contact surface 78 of the flexible skirt 80 into abutting contact with the patient's skin at the anchoring site surrounding the needle entry point, the common collar 152 is slidably advanced over the tubular body 86 to stiffen the flexible stabilization patch 142. Advancing the common collar 152 away from the flexible hub joint 116 allows the latter to bend under loads. Stiffening of the flexible stabilization patch 142 in cooperation with the flexible hub joint 116 of the hub 102 isolates the needle cannula 52 from external loads applied to the needle apparatus 150.

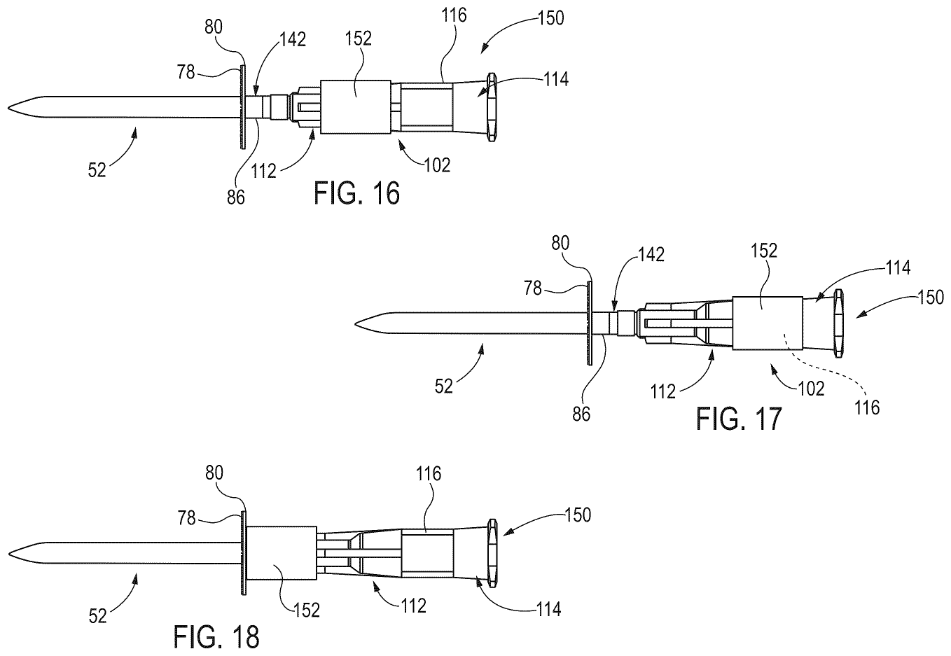

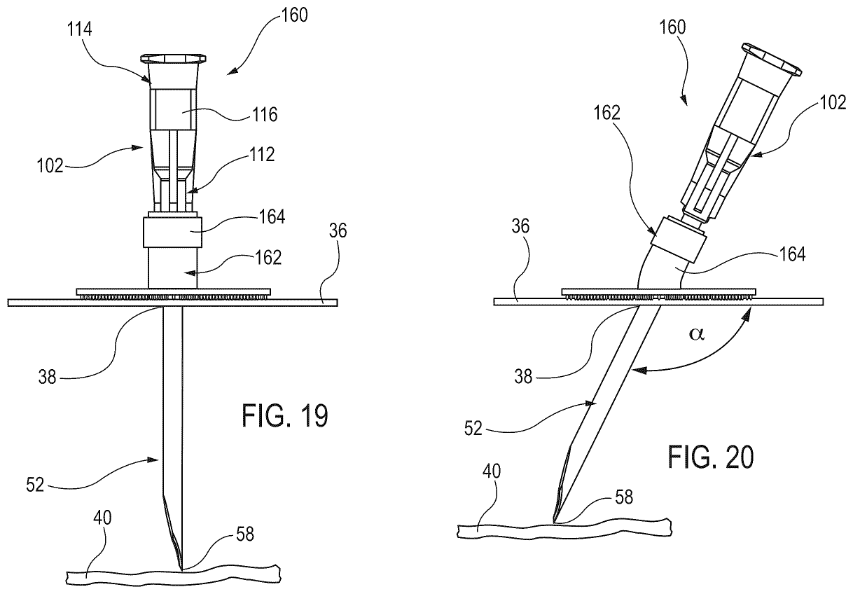

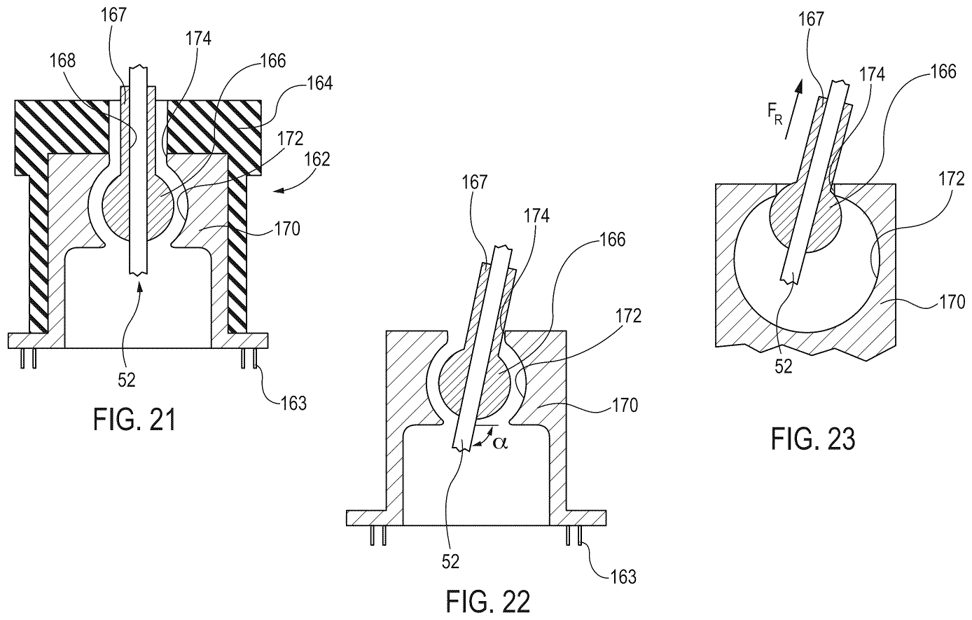

The needle apparatus 160 of FIGS. 19-23, like the needle apparatuses 140 and 150 incorporate a flexible stabilization patch. Needle apparatus 160 incorporates the needle cannula 52 and multi-piece flexible hub 102, with the distal segment 112, proximal segment 114 and intermediate hub flex joint 116 components of FIGS. 8-10. Needle apparatus 160 comprises a flexible ball-and-socket stabilization patch 162 with micro-needle anchoring media 163 disposed on its contact surface. A flexible cover 164 envelopes the ball-and-socket stabilization patch 162. Referring to FIGS. 19 and 21, a ball joint 166 includes a ball sleeve 167 and a through-bore 168, which captures the needle cannula 52. The needle 162 is coaxial with a socket joint 170. The socket joint 170 defines a socket cavity 172 that captures the ball joint 166 loosely therein, allowing free swiveling of the socket joint relative to the captured needle cannula 52 and the ball joint (see angle α in FIG. 22). A socket neck 174 is oriented on a proximal end of the socket joint 170 and allows the proximal end of the ball sleeve 167 to project out of the socket joint. The outwardly projecting portion of the ball sleeve 167 is coupled to the flexible cover 164. The socket neck 174 is dimensioned to retain the ball joint 166 therein to prevent relative axial separation of ball joint from the socket joint 170. Retraction of the flexible cover 164 and ball sleeve 166 in the direction F_{R} (see FIG. 23) locks the outer diameter of the ball into rigid contact with the socket neck 174.

As shown in FIG. 20, the needle apparatus 160 facilitates anchoring of the micro-needle anchoring media 163 to a patient's skin 36 even though the needle cannula 52 is oriented at an angle α on the penetration path between the needle entry point 38 to the penetration site 40. Prior to needle penetration, the ball-and-socket joint of the stabilization patch 162 is in an initial state shown in FIG. 21, wherein the contact surface and the stabilization needles are oriented perpendicular to the respective first and second central axes of the needle cannula 62 and the flexible needle hub 102. Referring to FIGS. 20 and 22, after advancement of the needle cannula 52 into the patient at the angle α, the socket joint 170 is swiveled to orient and abut the micro-needles 163 perpendicular to the patient's skin 36, thereby anchoring the stabilization patch 162 to the patient. Next, as shown in FIG. 23, the flexible cover 164 and its coupled ball sleeve 167 are retracted by exerting force F_{R} on them to lock the ball joint 166 within the socket neck 174. Before retracting the needle apparatus 160 from the patient, the flexible cover 164 and the ball sleeve 167 are advanced slightly to unlock the ball joint 166 from the socket neck 174, allowing the ball socket 170 to be separated from the patient's skin 36.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A medical needle apparatus comprising:
an elongated needle cannula defining a first central axis through a first lumen formed therein, a proximal first end, and a distal first tip for advancement into a body of a patient;
a needle hub defining a second central axis through a second lumen formed therein, a distal second tip of the hub coupled to the first end of the needle cannula and a proximal second end of the hub defining an open, exposed hub cavity that is in common fluid communication with the first and second lumens;
a stabilization patch defining a third central axis through a third lumen formed therein, the third lumen circumscribing an outer circumferential surface of the needle cannula, the stabilization patch having a contact surface on a distal third tip thereof for abutting contact with a selected external anchoring site of a patient's body that surrounds a needle entry point, which entry point initiates a penetration pathway of the needle cannula that terminates at a selected needle penetration site within the patient, and a proximal, third end facing the second end of the needle hub, the stabilization patch having an axial length between its third axial tip and its third axial end that is less than a corresponding axial length of the needle cannula, the stabilization patch selectively slidable bidirectionally along the needle cannula from the first distal tip towards the second end of the needle hub; and
the respective first, second and third central axes mutually coaxial with each other.

2. The medical needle apparatus of claim 1, wherein the contact surface comprises a distal surface of a pliable skirt that projects radially from the stabilization patch, perpendicular to the third central axis.

3. The medical needle apparatus of claim 2, the distal surface of the pliable skirt further comprises anchoring media selected from the group consisting essentially of molded pins, micro needles, or medical grade adhesive, or a suction cup.

4. The medical needle apparatus of claim 2, the stabilization patch further comprises:
a tubular body with a finger grasping surface proximate the third end thereof; and
the outer circumferential surface of the needle cannula forming a radially projecting rib proximate the first end for abutting and retaining engagement with a surface defining the third lumen when the tubular body is oriented in contact with the rib.

5. The medical needle apparatus of claim 1, the contact surface comprises a distal surface of a rigid flange that projects radially from the stabilization patch, perpendicular to the third central axis.

6. The medical needle apparatus of claim 5, the distal surface of the rigid flange further comprises anchoring media selected from the group consisting essentially of molded pins, or micro needles, or medical grade adhesive.

7. The medical needle apparatus of claim 5, the stabilization patch further comprises:
a tubular body with a finger grasping surface proximate the proximal end thereof; and
the outer circumferential surface of the needle cannula forming a radially projecting rib proximate the first end for abutting and retaining engagement with a surface defining the third lumen when the tubular body is oriented in contact with the rib.

8. The medical needle apparatus of claim 1, the hub cavity comprises a Luer connector.

9. The medical needle apparatus of claim 1, the needle hub further comprises a flexible hub with a flexible hub joint interposed between and coupled to the second tip and to the second end of the hub, the flexible hub joint transferring loads applied to the needle assembly that are aligned with the coaxial central axes, and the flexible hub joint isolating non-axially oriented deflection loads applied to the second end without deflecting the second tip or the needle cannula.

10. The medical needle apparatus of claim 1, further comprising a flexible stabilization patch joint that is interposed between and coupled to the contact surface and the third end of the stabilization patch.

11. The medical needle apparatus of claim 10, further comprises a shape-memory material flexible stabilization patch joint, which in a relaxed state defines a central through passage that is coaxial with the second central axis.

12. The medical needle apparatus of claim 11, further comprising a stabilization patch collar circumscribing the stabilization patch, selectively slidable axially to circumscribe the flexible stabilization patch joint and resist flexure deformation thereof.

13. The medical needle apparatus of claim 1, further comprising a ball-and-socket stabilization patch joint that is interposed between and coupled to the contact surface and the first end of the stabilization patch.

14. The medical needle apparatus of claim 13, ball-and-socket stabilization patch joint lockable in a selected angular orientation.

15. The medical needle apparatus of claim 13, further comprising a stabilization patch collar circumscribing the stabilization patch, selectively slidable axially to circumscribe the ball-and-socket stabilization patch joint and resist flexure deformation thereof.

16. The medical needle apparatus of claim 1, further comprising:
a flexible hub with a flexible hub joint interposed between and coupled to the second tip and to the second end of the hub, the flexible hub joint transferring loads applied to the needle assembly that are aligned with the coaxial central axes, and the flexible hub joint isolating non-axially oriented deflection loads applied to the second end without deflecting the second tip or the needle cannula; and
a flexible stabilization patch joint that is interposed between and coupled to the contact surface and the third end of the stabilization patch.

17. The medical needle apparatus of claim 16 further comprising at least one or more of:
a hub collar circumscribing the flexible hub, selectively slidable axially to circumscribe the flexible hub joint and resist non-axially oriented flexure deformation thereof; and
a stabilization patch collar circumscribing the stabilization patch, selectively slidable axially to circumscribe the flexible stabilization patch joint and resist flexure deformation thereof.

18. The medical needle apparatus of claim 16, further comprising a shared collar selectively slidable axially to circumscribe the flexible hub joint or the stabilization patch joint.

19. A method for treating a patient by advancing the medical needle apparatus of claim 1 into a selected needle penetration site within a patient, comprising:
selecting a needle penetration site of a patient;
orienting the first tip and first central axis of the needle cannula at a needle entry point, which entry point initiates a penetration pathway of the needle cannula that terminates at the selected penetration site;
advancing the needle cannula into the patient, along the penetration pathway, to orient the first tip within the selected penetration site; and
sliding the contact surface of the stabilization patch into abutting contact with the patient's body at an anchoring site surrounding the needle entry point, thereby coupling it to the patient's body, and when so coupled the stabilization patch resisting external deflectional loads applied to the needle apparatus.

20. The method for treating a patient of claim 19, further comprising after coupling the contact surface to the patient's skin:
coupling a syringe tip of a syringe to a Luer connector formed in the hub cavity; and
dispensing contents of the syringe to the patient's penetration site.

21. The method for treating a patient of claim 20, further comprising after dispensing the syringe contents to the patient's penetration site:
de-coupling of the contact surface of the stabilization patch from the patient's body by retracting it toward the needle hub; and
retracting the needle apparatus from the patient.

22. A medical needle apparatus, comprising:
an elongated needle cannula defining a first central axis through a first lumen formed therein, a proximal first end, and a distal first tip for advancement into a body of a patient;
a flexible needle hub defining a second central axis through a second lumen formed therein, a distal second tip of the hub coupled to the first end of the needle cannula and a proximal second end of the hub defining an open, exposed hub cavity that is in common fluid communication with the first and second lumens;
the first central axis and the second central axis coaxial with each other,
a flexible hub joint that is interposed between and coupled to the second tip and to the second end of the flexible needle hub, the flexible hub joint transferring loads applied to the needle assembly that are aligned with the coaxial central axes, and the flexible hub joint isolating non-axially oriented deflection loads applied to the second end without deflecting the second tip or the needle cannula.

23. The medical needle apparatus of claim 22, comprising a shape-memory material flexible hub joint, which in a relaxed state defines a central through passage that is coaxial with the second central axis.

24. The medical needle apparatus of claim 23, the flexible hub joint comprising a tubular construction.

25. The medical needle apparatus of claim 23, further comprising a hub collar circumscribing the flexible needle hub, the hub collar selectively slidable axially to circumscribe the flexible hub joint and resist non-axially oriented flexure deformation thereof.

26. The medical needle apparatus of claim 22, the flexible hub joint comprising a tubular construction.

27. The medical needle apparatus of claim 22, further comprising a hub collar circumscribing the needle hub, selectively slidable axially to circumscribe the flexible hub joint and resist non-axially oriented flexure deformation thereof.

28. The medical needle apparatus of claim 22, further comprising a stabilization patch defining a third central axis through a third lumen formed therein, the third lumen circumscribing an outer circumferential surface of the needle cannula, the stabilization patch having a contact surface on a distal third tip thereof for abutting contact with a selected external anchoring site of a patient's body that surrounds a needle entry point, which entry point initiates a penetration pathway of the needle cannula that terminates at a selected needle penetration site within the patient, and a proximal, third end facing the second end of the needle hub, the stabilization patch having an axial length between its third axial tip and its third axial end that is less than a corresponding axial length of the needle cannula, the stabilization patch selectively slidable bidirectionally along the needle cannula from the first distal tip towards the second end of the needle hub.

29. The medical needle apparatus of claim 28, further comprising a flexible stabilization patch joint that is interposed between and coupled to the contact surface and the first end of the stabilization patch.

30. The medical needle apparatus of claim 29, further comprising a stabilization patch collar circumscribing the stabilization patch, selectively slidable axially to circumscribe the flexible stabilization patch joint and resist flexure deformation thereof.

31. The medical needle apparatus of claim 30, further comprising a hub collar circumscribing the flexible needle hub, selectively slidable axially to circumscribe the flexible hub joint and resist non-axially oriented flexure deformation thereof.

32. The medical needle apparatus of claim 29, further comprising at least one or more of
a hub collar circumscribing the needle hub, selectively slidable axially to circumscribe the flexible hub joint and resist non-axially oriented flexure deformation thereof; and
a stabilization patch collar circumscribing the stabilization patch, selectively slidable axially to circumscribe the flexible stabilization patch joint and resist flexure deformation thereof.

33. The medical needle apparatus of claim 29, further comprises a shared collar selectively slidable axially to circumscribe the flexible hub joint or the stabilization patch joint and resist flexure deformation of the circumscribed joint.
